(19) United States
(12) Patent Application Publication
(10) Pub. No.: US 2006/0094998 A1
(43) Pub. Date: May 4, 2006

(54) Vascular access device anchor and occlusive dressing

(76) Inventor: Lisa A. S. Gorton, Ware, MA (US)

Correspondence Address: Lisa A. S. Gorton, 34 West Main Street, Ware, MA, 01082, US

(21) Appl. No.: 10/980,228

(22) Filed: Nov. 4, 2004

(51) Int. Cl. A61F 15/00; A61F 13/00

(52) U.S. Cl. 602/42; 602/47

(57) ABSTRACT

An anchoring and occlusive dressing made of a thin polymeric and adhesive material (Class 602, Subclass 42, 52, and 58) in a three dimensional, upside-down “T” shape. The broad, flat area (top of the “T”) can be of varying shapes and sizes with an opening approximately two-thirds the width of the dressing at its widest part. The stem of the “T” is made up of two parallel pieces of the polymeric and adhesive material that are formed perpendicular to the base, but are flexible and will follow the movement of the access device. The parallel pieces are adhesive on the sides that are facing each other with a small area at the top, about 0.3-0.5 inches in height, that is reinforced and non-adhesive. This system allows the total occlusion of the insertion site, not currently accomplished with standard IV dressings, thereby eliminating the open path that allows bacteria access to the site. With the use of skin prep to increase the adhesion, and antibacterial material around the insertion site, the dressing can remain intact for up to 7 days without increased risk of infection. This dressing can be used for IV insertion sites of all sizes and sites, arterial infusion sites, interventional catheter sites, nephrostomy and other drainage tube sites.

### BACKGROUND OF INVENTION

Flat, transparent, polymeric dressings have been used for years in the healthcare industry. They allow visualization of the insertion site while helping to prevent environmental contamination of the insertion wound. The problem has been that it is impossible to completely seal off the wound from contaminants because it is not possible to seal around the catheter itself. The “tunnel” around the tubing provides a direct path to the wound that contaminants can travel and infect the site. Also catheters tend to move and shift around which increases the size of the “tunnel” by increasing the size of the area not adhered around the catheter. Often it is necessary to reposition the catheter for better flow. This shortens the safe time between dressing changes and increases the discomfort of frequent dressing changes and the risk of infections. Currently site dressing needs to be removed and reapplied thus exposing insertion site to contamination.

This dressing allows for total occlusion around the catheter, and a fair amount of lateral movement of the catheter without disturbing the integrity of the dressing, thereby extending the life of each dressing and decreasing the risk of contamination of the insertion site. The dressings come in different sizes and shapes allowing the healthcare professional to choose which will provide the appropriate fit for the situation, and the better the fit the more effective the dressing is at remaining intact and preventing contamination.

### BRIEF DESCRIPTION OF DRAWINGS

FIG. 1 is a view looking directly down from the top at the dressing.

FIG. 2 is a view looking at the lateral view of the long side of dressing.

FIG. 3 is a view looking at the lateral view of the short side of the dressing.

FIG. 4 is a view looking at the dressing from above and off to the side, including the catheter in place.

FIG. 5 is a view of the specially designed non-adhesive coated protective paper used as the tab between the sleeves. The shaded area is the uncoated side, the non-shaded areas at either end are the non-adhesive coated side. This tab is described in claims section, 8.

FIG. 6 is a lateral view of the dressing applied to a curved skin surface, including the catheter in place.

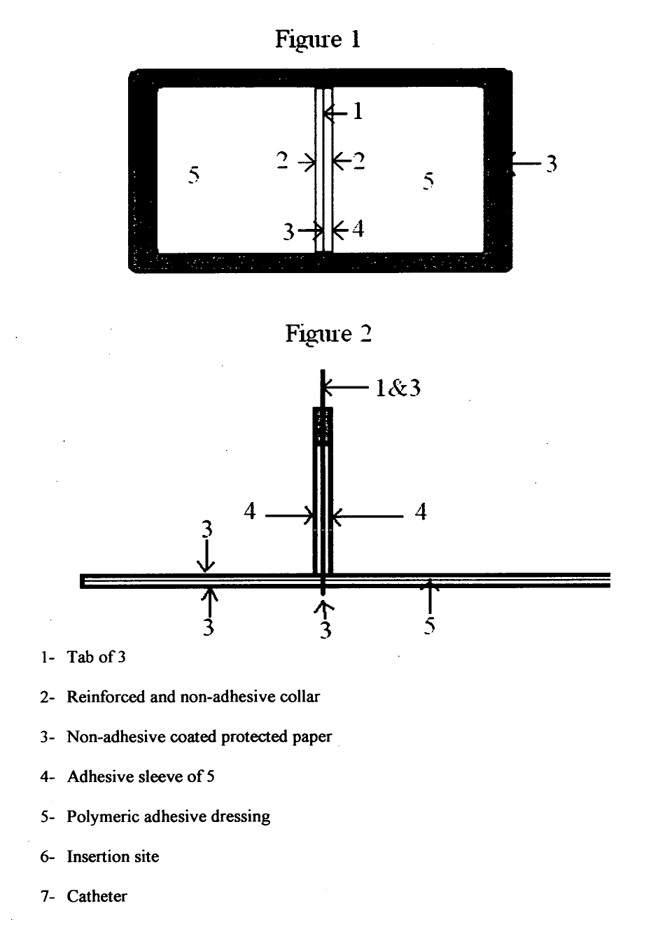

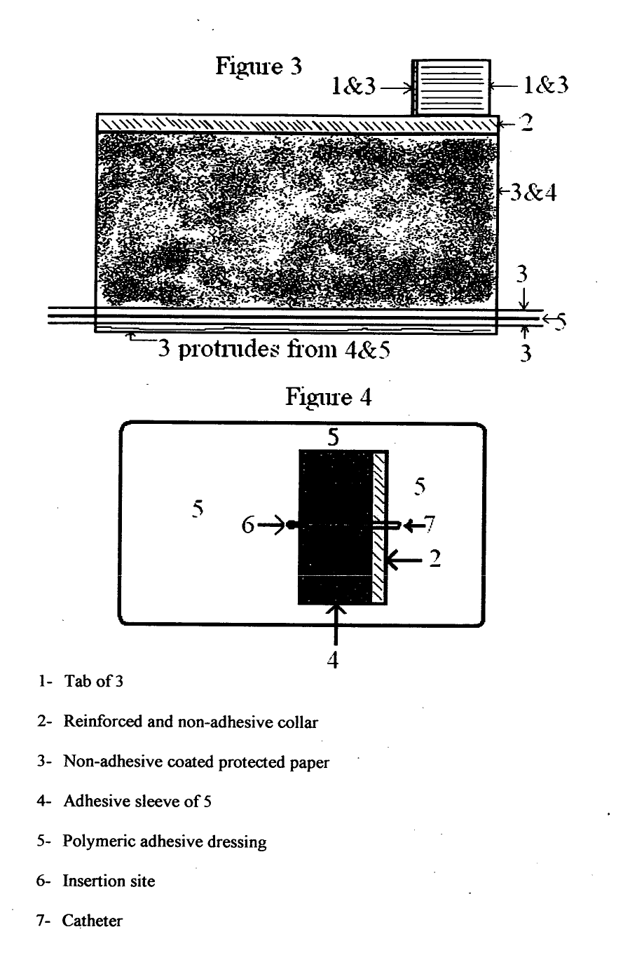

In FIGS. 1-4 the number key is:
1—Tab of non-adhesive coated protective paper between sleeve, non-adhesive side toward adhesive side of polymeric dressing
2—Reinforced and non-adhesive collar area of the sleeve
3—Non-adhesive coated protective paper
4—Adhesive sleeve
5—Polymeric adhesive dressing
6—Insertion site
7—Catheter

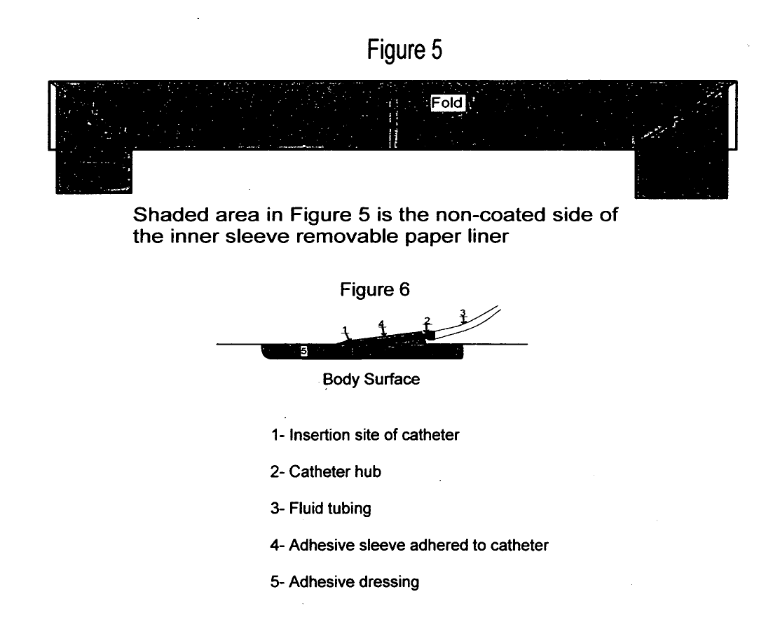

In FIGS. 5-6 the number key is:
1—Insertion site of catheter
2—Catheter hub
3—Fluid tubing
4—Adhesive sleeve adhered to catheter
5—Adhesive dressing

What is claimed is:

1. An anchoring and occlusive dressing which allows total occlusion and securing of a wide variety of catheters, allowing them some freedom of movement and with proper preparation, the dressing can remain in place for 7 days without risk of infection.

2. An anchoring and occlusive dressing as in claim 1, wherein a polymeric and adhesive dressing in a 3 dimensional “T” shape is made of one sheet of polymeric material.

3. An anchoring and occlusive dressing as in claim 1, wherein a polymeric and adhesive dressing in a 3 dimensional “T” shape where the broad, flat area (top of the “T”) can be of varying shapes and sizes and the stem of the “T” is approximately 1.5 inches in height and two-thirds the width of the dressing.

4. An anchoring and occlusive dressing as in claim 1, wherein a polymeric and adhesive dressing in a 3 dimensional “T” shape, where the broad, flat area (top of the “T”) is coated in adhesive and attached to a non-adhesive coated protective paper which is split in the center of the dressing.

5. A non-adhesive coated protective paper as in claim 4, wherein a split occurs in the center of the shorter side for easy separation and removal from the polymeric dressing once attached to the skin.

6. An anchoring and occlusive dressing as in claim 1, wherein the non-adhesive side of the broad, flat area (top of the “T”) is attached to a non-adhesive coated protective paper around the outside of the top one-sixth of the polymeric dressing, providing support and stability after bottom layer is removed prior to application to the skin.

7. An anchoring and occlusive dressing as in claim 1, wherein the stem of the “T” (sleeve) consists of two parallel pieces of the polymeric and adhesive material that are perpendicular to the base, ending in an area approximately 0.3-0.5 inches in height, that is reinforced and non-adhesive.

8. The two parallel pieces of the polymeric and adhesive material as in claim 7, wherein are separated by a specially designed non-adhesive coated protective paper that protrudes through the non-adhesive protective paper in claim 3.

9. The specially designed non-adhesive coated protective paper as in claim 8, wherein a straight strip is folded in half with the coated side facing out, then each end is folded towards the center at a 180 degree angle, then folded at a 45 degree angle so that the coated sides within that fold are facing each other and leaving an approximately 0.5 inch tab on both ends rising above the reinforced non-adhesive area of the polymeric dressing.

10. A method for applying the anchoring and occlusive dressing to the patient, the method comprising the steps of:
Using sterile technique throughout procedure
Open package of dressing, maintaining sterility of dressing
Prepare insertion site and surrounding area by cleaning with oil removing solution, such as rubbing alcohol.
Clean around insertion site and surrounding area with antibacterial/bactericidal solution
Apply skin protectant to the area that the dressing will be adhering to, allow to become tacky
Suggested application of local bactericidal material at insertion site
Open hole through sleeve by applying pressure on the ends of the sleeve and pass end of catheter through the opening
Position dressing so that the lie of catheter is perpendicular to the wide length of the sleeve and the dressing is flush to the skin
While holding the catheter in the desired position, through the sleeve of the dressing, lift up one side of the base, peel away the non-adhesive protective paper, apply the adhesive side to the skin and repeat for the other side.
Gently remove the non-adhesive protective paper in the sleeve using the tabs while holding the catheter in place just above the top of the sleeve
Lay the catheter in position and seal the sides of the sleeve together around the catheter
Gently remove the top layer of non-adhesive protective paper from around the dressing using the split as a tab
Label dressing with date, time, and initials
Any extra length of catheter can be secured using tape if needed

11. When dressing needs to be removed:
Using clean technique
Peel base of dressing completely off skin
Using reinforced non-adhesive area at the top of the sleeve, separate the sleeve and carefully remove from catheter
